(19) United States
(12) Patent Application Publication
(10) Pub. No.: US 2021/0358639 A1
(43) Pub. Date: Nov. 18, 2021

(54) Pandemic Health Risk Management System and Method to Accelerate Voluntary Notification to Trace Contacts and to Encourage Self-Quarantine

(71) Applicant: Michael A. Long, Rolling Hills Estates, CA (US)

(72) Inventor: Michael A. Long, Rolling Hills Estates, CA (US)

(21) Appl. No.: 16/872,385

(22) Filed: May 12, 2020

(51) Int. Cl. G16H 50/80; G16H 10/20; G06Q 50/00; G16H 10/60; G06Q 10/10; G06Q 10/08; G06Q 30/06; G06Q 50/26; G06Q 40/08; G16H 40/20; G06Q 30/00; H04L 9/32; H04L 9/06; H04L 29/08; H04L 12/58

(52) U.S. Cl. CPC G16H 50/80; H04L 2209/38; G06Q 50/01; G16H 10/60; G06Q 10/109; G06Q 10/083; G06Q 30/0633; G06Q 50/26; G06Q 40/08; G16H 40/20; G06Q 30/0185; H04L 9/3268; H04L 9/0637; H04L 67/18; H04L 51/046; G16H 10/20

(57) ABSTRACT

A system to manage pandemic health risk provides a pandemic app having a user interface that runs on a mobile device, a voluntary participant can register to use the pandemic app, and a trace contact is identified in the pandemic app by the participant through the user interface.

### BACKGROUND OF THE INVENTION

### Field of Invention

The present invention relates to pandemic health risks and contact tracing.

### 2. DESCRIPTION OF THE RELATED ART

The COVID-19 pandemic has rocked the entire world. Contact tracing is limited in its current reach. No widely adopted tool exists that has yet been adopted by the public to quickly and voluntarily assist in self-quarantine efforts amongst individuals and their contacts. Instead, convention follows orders of state governors in parental manner, causing resentment and significant resistance to advice from medical professionals to encourage self-quarantine during the pandemic.

Some highlights of recent history relating to COVID-19 include the following events:

On Mar. 11, 2020, the World Health Organization declared COVID-19 a pandemic.

On Apr. 27, 2020, health experts requested $46.5 billion to hire contact tracers to address the COVID-19 pandemic. A priority need of utmost importance exists and subsists.

On May 1, 2020, the TRACE Act was proposed in the House of Representatives to fund $100 billion to promote contact tracing, specifically, “To authorize the Secretary of Health and Human Services to award grants to eligible entities to conduct diagnostic testing for COVID-19, and related activities such as contact tracing, through mobile health units and, as necessary, at individuals' residences, and for other purposes.”

It is known that people do not wish to be corralled and have their personal freedoms impinged by government. For example, a large public protest in Huntington Beach, Calif. demonstrated the importance of individuals to have a choice. Therefore, personal choice and the ability to decide is paramount to effective pandemic risk management.

Software as a Medical Device (SaMD) are known. The Food and Drug Administration (FDA) recognizes certain software as a medical device (SaMD) including but not limited to: mobile apps to help diagnosis, prevention, monitoring, treatment or alleviation of a disease (quarantine); and SaMDs can be capable of running on non-medical device platforms. International Medical Device Regulators Forum (IMDRF) 9 Dec. 2013, 5.1 and 5.2.1.

Known in the related art is U.S. Pat. No. 8,405,503 to Wong for “System and method for creating a proximity map of living beings and objects” filed on Sep. 14, 2009. Wong teaches that each person is to have a proximity-sensing device with unique identifier which is recorded by encountered units to map contacts for later retrieval at a given site. Wong does not teach or suggest a system for participants to voluntarily encourage self-quarantine to trace contacts to manage pandemic health risk.

Known in the related art is U.S. Pat. No. 8,335,298 to Clawson for “Pandemic diagnostic and intervention tool for emergency dispatch” also filed on Sep. 4, 2009. Clawson discloses a diagnostic tool for an emergency dispatcher responding to emergency calls. Clawson does not teach or suggest having participants to voluntarily assist trace contacts in self-quarantine to manage pandemic health risk.

Known in the related art is U.S. Pat. No. 7,993,266 for “Early detection of contagious diseases” to Colston et al. filed on Feb. 26, 2003 claiming benefit of provisional application No. 60/375,986 filed on Apr. 26, 2002. Colston et al. discloses an electronic proximity apparatus and a surveillance method using such an apparatus for alerting individuals that are exposed to a contagious disease. Colston et al. does not teach or fairly suggest a system for participants to voluntarily encourage self-quarantine to trace contacts to manage pandemic health risk.

Known in the related art is Application No. 20060220879 for “Apparatus and method of acquiring and storing data of close contacts” to Chan. Chan discloses stored identity details to determine identities of persons with whom close contact had been made within a predetermined time. Chan does not teach or suggest a participant sending a message to a trace contact to advise or encourage self-quarantine.

It can be seen then, that there is a need for a system to address the foregoing needs and to provide a technology that is capable of efficiently curbing the spread of infection by notifying and encouraging self-quarantine. It can also be seen that there is a need for such a s system to provide opt-in means to willing participants that desire to promote health and safety for themselves, their loved ones and their community. Furthermore, it can be seen that there is a need to address any combination of the foregoing needs alone or in combination.

### SUMMARY OF THE PRESENT INVENTION

The present invention seeks to resolve the shortcomings of conventional and parental systems by providing a voluntary system that a participant can willingly provide information to encourage others, including trace contacts, to self-quarantine.

The present invention provides a system to manage pandemic health risk by and through a web accessible app having a user interface that runs on a mobile device, a voluntary participant can register to use the pandemic app, and a trace contact is identified in the pandemic app by the participant through the user interface.

An aspect of the present invention is to address the foregoing needs and to provide a technology that is capable of efficiently curbing the spread of infection by notifying and encouraging self-quarantine.

Another aspect of the present invention is to provide opt-in means to willing participants that desire to promote health and safety for themselves, their loved ones and their community.

### BRIEF DESCRIPTION OF THE DRAWINGS

Non-limiting exemplary embodiments are provided in the drawings as follows:

FIG. 1 is an overall view of an embodiment of the present invention;

FIG. 2 is an overall view of an embodiment of the present invention;

FIG. 3 is an overall view of an embodiment of the present invention.

FIG. 4 is an overall view of an embodiment of the present invention;

FIG. 5 is an overall view of an embodiment of the present invention;

FIG. 6 is an overall view of an embodiment of the present invention.

FIG. 7 is an overall view of an embodiment of the present invention;

FIG. 8 is an overall view of an embodiment of the present invention;

FIG. 9 is an overall view of an embodiment of the present invention.

FIG. 10 is an overall view of an embodiment of the present invention;

FIG. 11 is an overall view of an embodiment of the present invention;

FIG. 12 is an overall view of an embodiment of the present invention.

FIG. 13 is an overall view of an embodiment of the present invention;

FIG. 14 is an overall view of an embodiment of the present invention; and

FIG. 15 is an overall view of an embodiment of the present invention.

### DETAIL DESCRIPTION OF THE PRESENT INVENTION

In the following description of the preferred embodiments, reference is made to the accompanying drawings that form a part hereof, in which is shown by way of illustration specific embodiments in which the invention may be practiced. It is to be understood that other embodiments may be utilized and structural changes may be made without departing from the scope of the present invention.

References throughout the specification to “interesting embodiment”; “possible embodiment”; “preferred embodiment”; “some embodiments”; “an embodiment”; and like reference to “embodiment” are non-limiting examples to aid in understanding the present invention. An “embodiment” provides that there can be one or more embodiments that can involve the given element or aspect of the invention. Thus, multiple instances of “an embodiment” and like reference do not necessarily refer to the same embodiment.

This specification provides for specific meanings with respect to the present invention, the meanings of which shall be understood as follows:

“Participant” and “voluntary participant” are interchangeable for purposes of this specification and can refer to a computing device, mobile device, downloaded app, or browser accessing the pandemic app of the present invention disclosed herein.

Any numbered “tier of trace contacts” can refer to groups of trace contacts that stem from a parent contact. In the case of initially identified trace contacts by a participant, the given n-tier of trace contacts input and identified by the participant is referred to as the first tier of trace contacts 202 as used herein. When a particular trace contact 200 later joins, in view of the participant's prior trace contact disclosures, second tier of trace contacts 204 then is understood because it is a degree of relationship removed from first tier of trace contacts 202 relative to participant 300. There can be overlapping trace contacts in any given tier because tiers need not be mutually exclusive for purposes of tiers of trace contacts as described herein. In other words, trace contact 200 can be in more than one tier depending on how the trace contact is reported.

An overarching principle to be understood in this specification is that the point of the present invention is to facilitate participation by the public in the interest of public health and safety balanced and tempered by the risk of public resistance and outcry that results from overly parental systems.

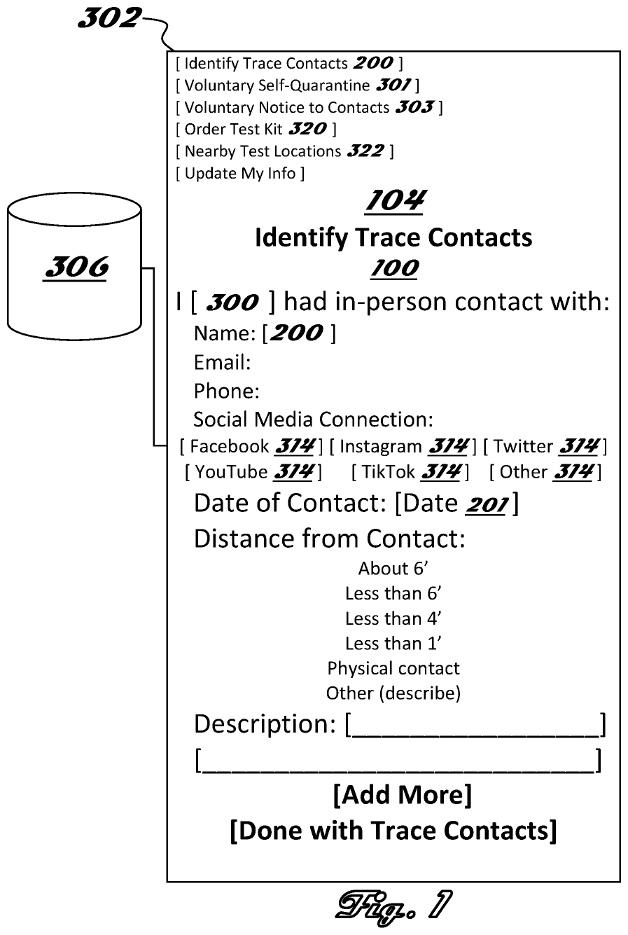

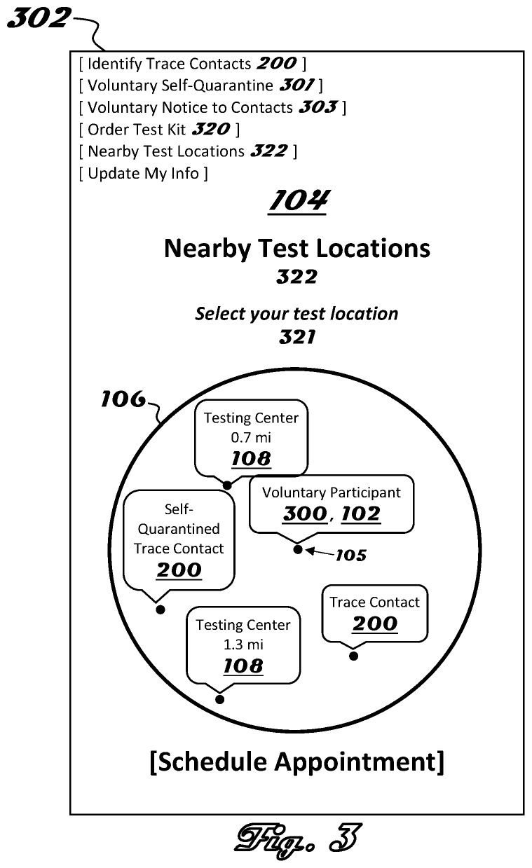

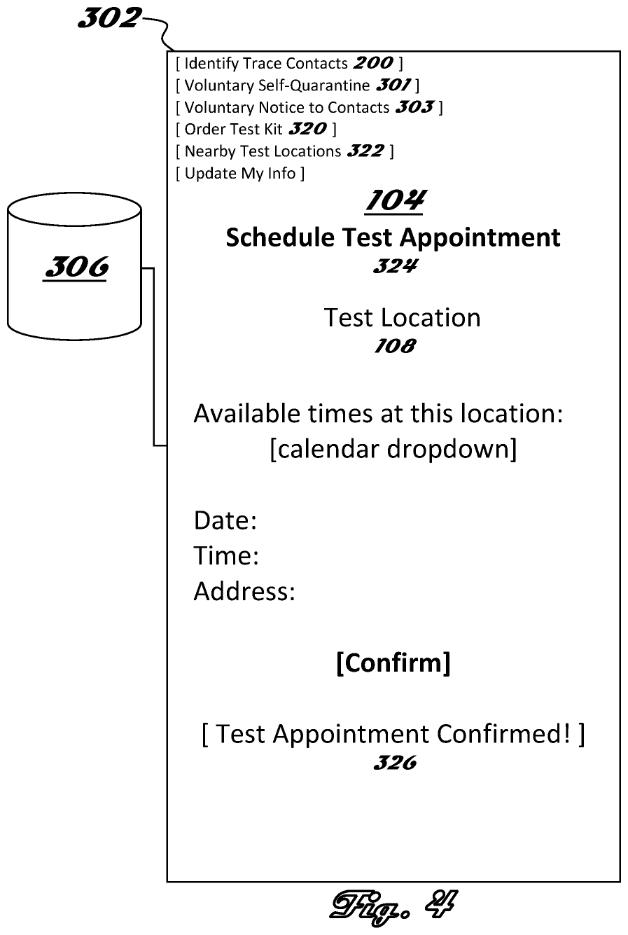

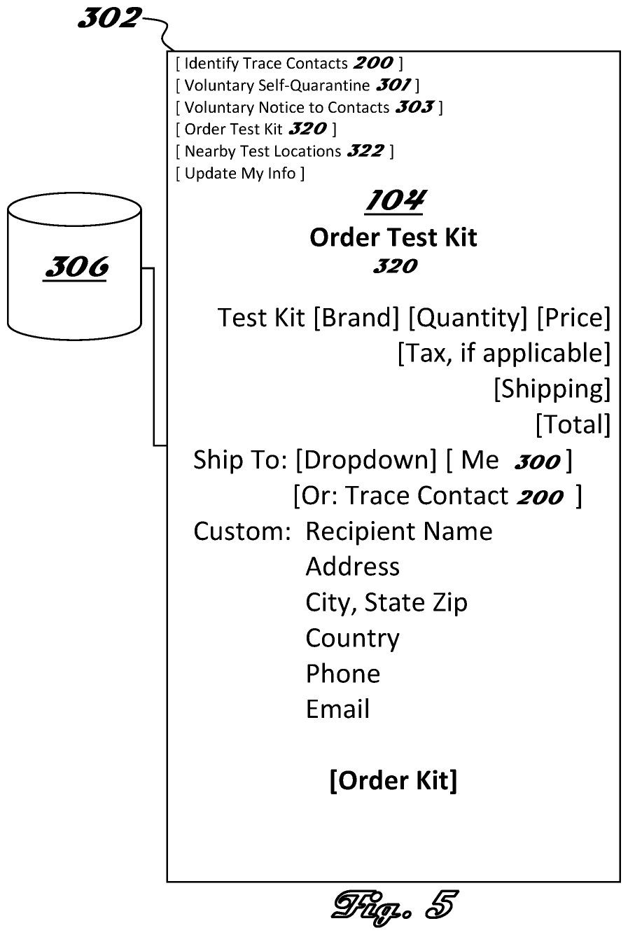

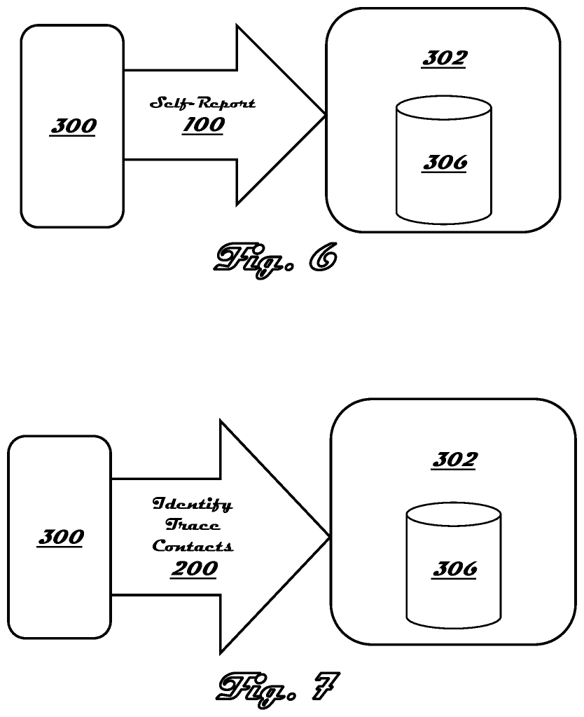

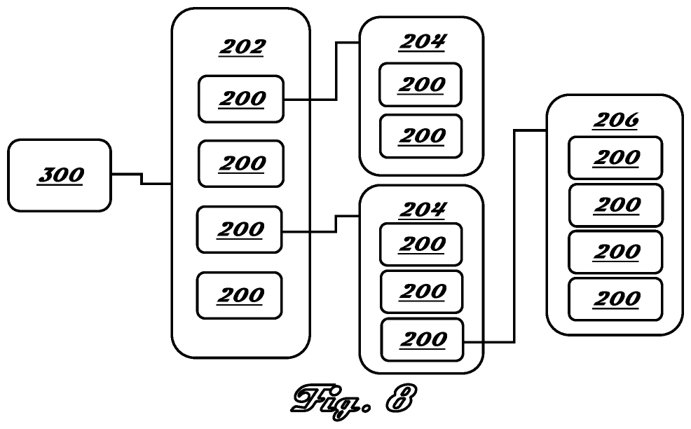

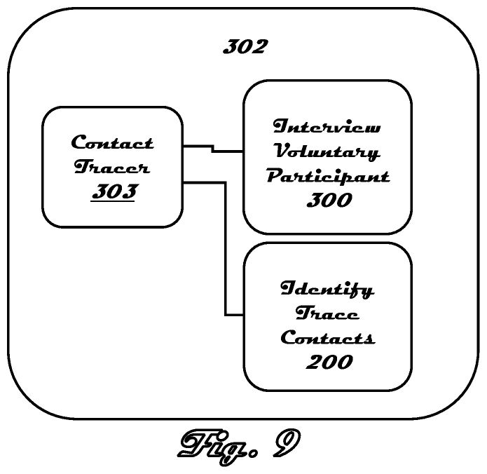

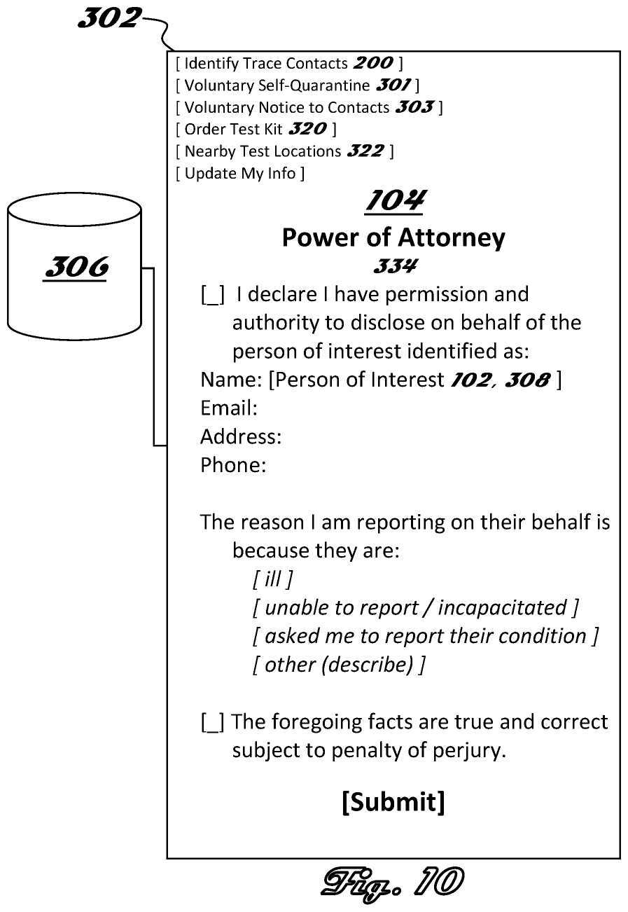

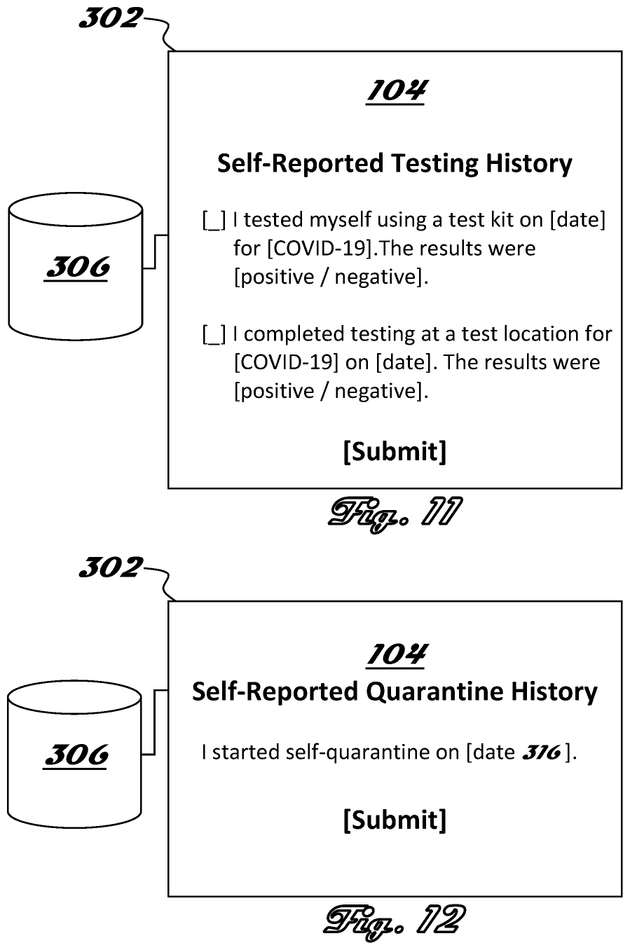

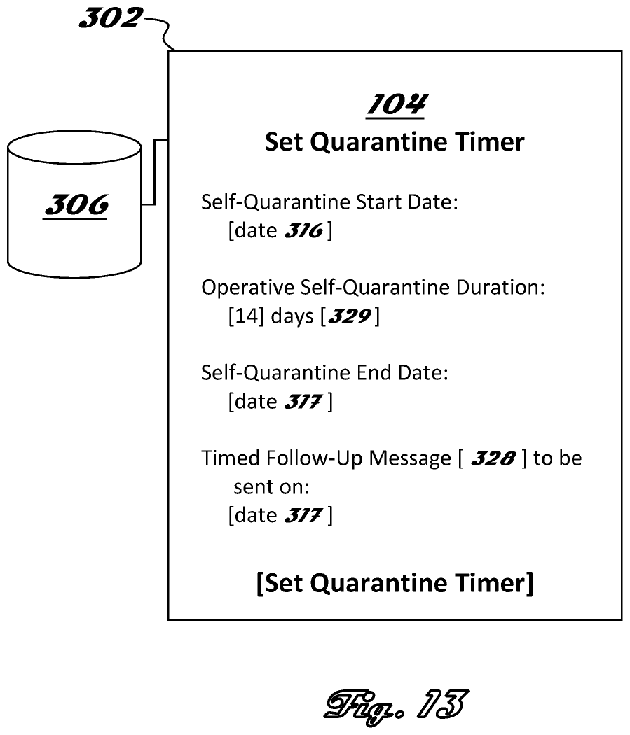

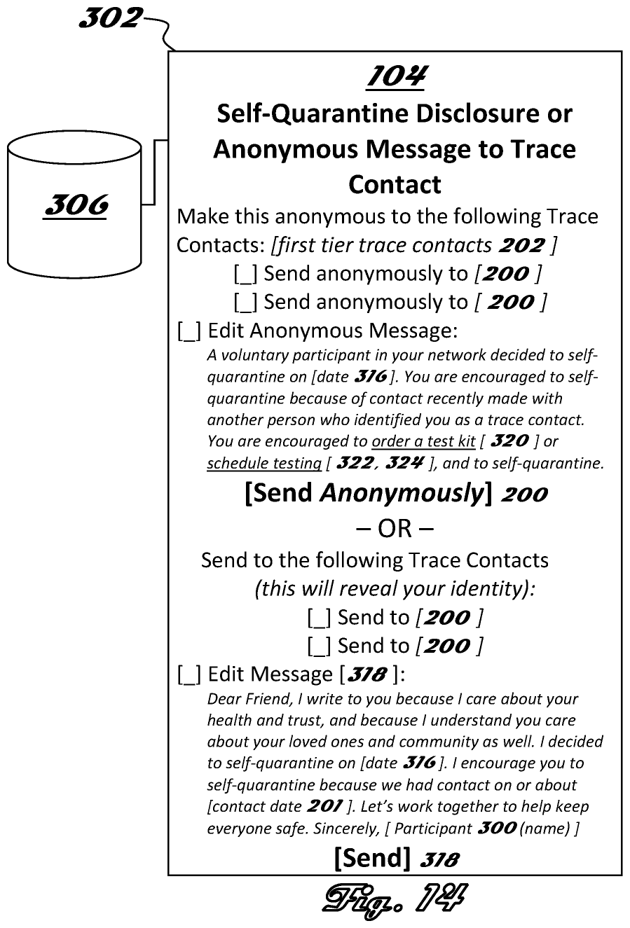

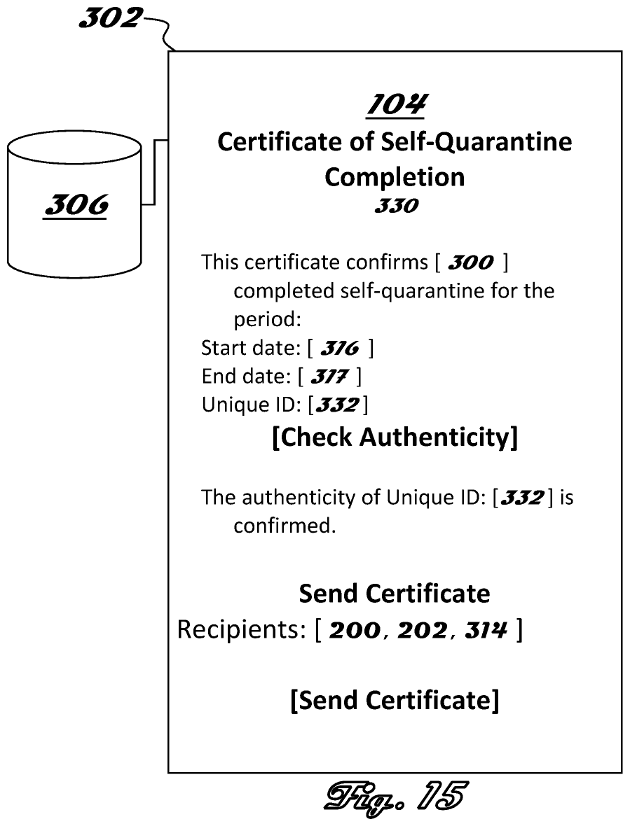

FIGS. 1-15 show possible embodiments of the present invention.

A system to manage pandemic health risk can have:

Identify trace contacts input form 100 can confirm participant 300 had contact with trace contact 200. Participant can voluntarily self-report to have infection.

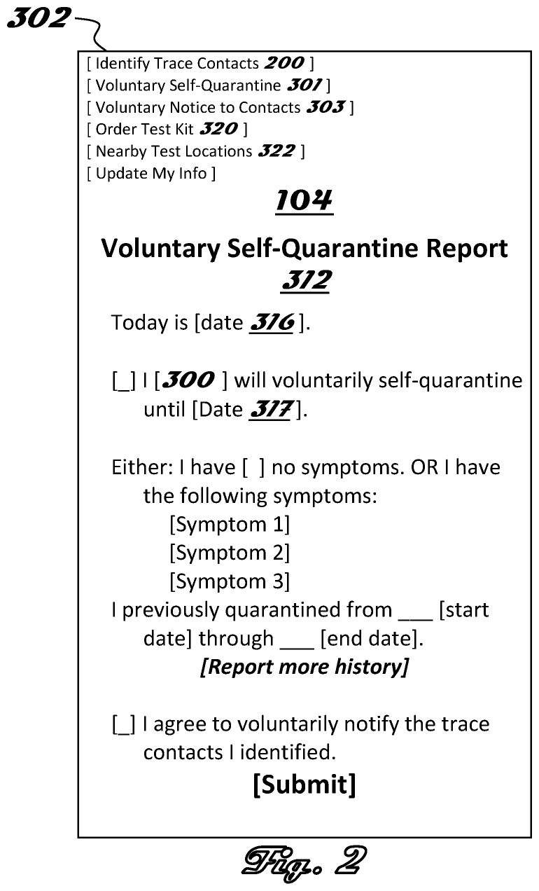

Voluntary self-quarantine report 312 (FIG. 2) can be generated, preferably on a given date of contact 316, by the participant 300 self-reporting to disclose that participant 300 has a symptom of the current pandemic concern or simply that the participant 300 will voluntarily self-quarantine due to contact. Prior history can be disclosed in the voluntary self-quarantine report 312. An end date can be set for the voluntary self-quarantine period 317.

Person of interest 102 can voluntarily become participant 300 in the pandemic app 302.

User interface 104 (FIGS. 1-5 and 10-15) can provide a graphical user interface for pandemic app 302 (FIGS. 1-15 throughout). User interface 104 can provide an intake process for participant 300 to input trace contacts 200 including name and contact information, such as social media username and platform, email, address, phone. User interface 104 can show geolocation 105 of identifier 108 for person of interest 102. user interface 104 can show map 106, testing location 108, trace contact 200 and participant 300. User interface 104 can provide scheduling function to set a date and time for testing at a test location 108. User interface 104 can provide ordering a test kit to be sent to participant 300. User interface 104 can provide ordering a test kit to be sent to trace contact 200. User interface 104 can provide an input screen to permit entry of trace contact information, social media, email, mobile phone number, and/or address of trace contacts 200 associated with participant 300. User interface 104 can provide an option for participant 300 to authorize pandemic app 302 to contact trace contacts 200. User interface 104 can display proximate test locations 108 on map 106. User interface 104 can provide an input to search for nearby test locations 108. Nearby test locations 322 can prompt to choose selected test location 321.

Trace contact 200 can be identified by participant 300 in pandemic app 302. Trace contact 200 can be within first tier of trace contacts 202. Trace contact 200 can be associated with person of interest 102 or participant 300. Trace contact 200 can be a known associate of the person of interest 102 or participant 300. Trace contacts 200 can provide a set of known associates of contacts can have known history concerning the subject of the instant pandemic, such as COVID-19. There can be a first and second tier of trace contacts 202. Trace contact 200 can voluntarily join pandemic app 302. Trace contact 200 can designate voluntary self-quarantine in response to a disclosure of infection from an earlier participant 300. There can be an at least one trace contact 200.

First tier of trace contacts 202 can be associated with person of interest 102 or participant 300. First tier of trace contacts 202 can have one or more trace contacts 200.

Second tier of trace contacts 204 can be associated with person of interest 102 through first-tier trace contact 200. Second tier of trace contacts 202 can have one or more trace contacts 200. Second tier of trace contacts 204 can be associated trace contact 200.

Third, fourth, or any number of nth tiers of trace contacts 206 can be exhaustively iterated to address pandemic concerns within pandemic app 302.

Participant 300 can be a voluntary user in pandemic app 302. Participant 300 can be person of interest 102. Participant 300 can designate trace contact 200 with whom the participant had recent contact in tracing disclosure record 306. Participant 300 can opt-in to voluntarily participate via pandemic app 302. Participant 300 can designate voluntary self-quarantine.

Voluntary self-quarantine 301 can be initiated by participant 300 as reported into pandemic app 302.

Pandemic app 302 can be a mobile application, social media or website any of which can be accessible through a web browser or with a downloadable app that runs natively on a mobile device. Pandemic app can have a database of registered participants 300. Pandemic app 302 can provide user interface 104. Pandemic app 302 can be used by voluntary participants 300 rather than as a parental tool to impose control on the population. One having an ordinary level of skill in the pertinent art would know how to incorporate pandemic app with the features of the present invention. Pandemic app 302 can provide for registration and secure authentication of person of interest 102, participant 300, trace contact 200 and tracer 304. Pandemic app 302 can permit authorized disclosure of conditions for which participant 300 has personal knowledge and medical authority, such as power of attorney, to disclose on behalf of a third person 308, such as person of interest 102 in situations where person of interest 102 is incapacitated or having severe symptoms that preclude them from logistically using pandemic app 302. Pandemic app 302 can provide a map and geolocations of participant 300 and trace contact 200. Once trace contact 200 signs onto pandemic app, then a second tier of trace contacts can be generated based on disclosures of subsequent trace contacts 200 by the initial trace contact 200 who becomes a voluntary participant 300. Pandemic app 302 can provide an option for participant 300 or trace contact 200 can request a test kit 320 to be shipped via a prompted message via a secure sockets layer URL to initiate an order to ship test kit 320 to trace contact 200.

Voluntary notification to one or more trace contacts 303 can be initiated by participant 300 as reported into pandemic app 302.

Tracer 304 can interview participant 300 via pandemic app 302. Tracer 304 can designate trace contact 200 with whom participant 300 had recent contact and can store trace contact association with participant 300 in tracing disclosure record 306.

Tracing disclosure record 306 can store identified tracing contacts 200. Tracing disclosure record 306 can store associations of each contact with a given participant 300 including date of contact 307 and date entered in pandemic app 309. Tracing disclosure record 306 can have subsequent geolocations associated with first tier of trace contacts 202. Tracing disclosure record 306 can be stored in a relational database.

Medical history 310 can be associated with person of interest 102. Medical history 310 can be associated with a participant 300. Medical history 310 can be voluntarily disclosed. Medical history 310 can be stored securely and can be HIPAA compliant.

Self-reported testing history report 311 can be provided by authorization of participant 300. Participant's self-reported testing history report 311 can be sent to trace contacts 200 of participant 300.

Voluntary self-quarantine report 312 can be provided by authorization of participant 300. Participant's self-reported quarantine history report 312 can be sent to trace contacts 200 of participant 300.

Social contacts 314 of participant 300 can be selected from: social media connections, email connections, and numeric phone contacts. Trace contacts 200 can be selected from social contacts 314 via social media platform, e.g., Facebook, Instagram, Twitter, Youtube, Snapchat, Tiktok, LinkedIn, and other social media platforms.

Initial date of quarantine 316 can be input by participant 300 or contact tracer 304 via user interface 104 of pandemic app 302.

Timer 317 can be associated with a first time of reporting to expire upon conclusion of a known duration 318. Timer 317 can be set to at least two weeks from the date of contact with person of interest 102 or participant 300. Timer 317 can be set for trace contact 200.

Self-quarantine message 318 can be sent to trace contact 200. Self-quarantine message 318 can be sent to trace contact 200. Self-quarantine message 318 can be sent to first tier of contacts 202 associated with participant 300. Self-quarantine message 318 can be sent to second tier of contacts 204 associated with each of trace contacts 200 in first tier of contacts 202. Self-quarantine message 318 can be sent to nth tier of contacts 204 associated with each of trace contacts 200 in n−1 tier of trace contacts. Self-quarantine message 318 can disclose the voluntary decision to self-quarantine of participant 300 and to notify trace contacts 200. Self-quarantine message 318 can disclose trace contact 200's voluntary decision to notify further trace contacts 200 and to self-quarantine. Self-quarantine message 318 can provide an option to order test kit 320. Self-quarantine message can provide an option to schedule test appointment 324. Message 318 to trace contact 200 can be from anonymous sender 319 via pandemic app 302. Message 318 can be issued via blockchain certificate and can be verified as to its authenticity by blockchain audit. Thereby, blockchain-based message 318 can reduce risk of spoofing or man-in-the-middle attacks.

Anonymous sender 319 can protect the identity of participant 300 or person of interest 102. Anonymous sender 319 can replace the identity of a sender of message 318 disclosing voluntary self-quarantine or voluntary self-testing. A benefit of anonymous sender is to reduce the social awkwardness of disclosing to certain select contacts or to reduce risk of retaliation while protecting public interest in managing health and safety of others.

Test kit 320 request can be sent to trace contact 200. Test kit 320 request can be sent to first tier of contacts 202 associated with participant 300. Test kit 320 can be paid for at least in part by governmental agency, healthcare insurance provider, participant, trace contact, or sponsor.

Selected test location 321 can be provided via user interface 104. Selected test location 321 can input the participant's geolocation to begin scheduling test appointment 324.

Nearby test locations 322 can show test locations 106 on map 106. Search result can be centered on the geolocation of trace contact 200 or participant 300. A closest distance option can be presented in timed follow-up message 328. Nearby test locations 322 can show participant 300 and relative proximate test locations 108 including distance, rating, and comments.

Schedule test appointment 324 can provide the step of arranging an appointment at a specific test location 108 for a given participant 300 or trace contact 200. Test locations 108 can be registered with pandemic app 302. Test locations 108 can provide scheduling functions to permit intake of patients, including to follow social distancing protocols to seek to minimize risk of cross-infection or exposure amongst those tested at a given test location 108.

Scheduled appointment 326 can provide date, time and test location 108. Scheduled appointment 326 can be provided to trace contact 200 to conduct local testing.

Timed follow-up message 328 can be sent to participant 300 at an end time of the contagious period for follow-up. Timed follow-up message 328 can be sent to trace contact 200 at an end time of the operative contagious period (or recommended quarantine period) 329 for follow-up. Timed follow-up message 328 can facilitate checking status to determine further steps following the quarantine. Further reporting can be performed with participant 300 and/or trace contacts 200.

Certificate of quarantine completion 330 can be generated. Certificate of quarantine completion 330 can have a unique identifier 332 and can be generated via blockchain. Certificate of quarantine completion 330 can be based on the voluntary self-reporting of a given participant 300 that was previously a newly entered trace contact 200. Certificate 330 can be securely issued, exchanged and verified via certificate server 334.

One having ordinary level of skill in the pertinent art would know how to incorporate the elements of the present invention to enable its use. One having ordinary level of skill in the pertinent art would know how to make and use the invention based on the disclosure of this specification.

### CONCLUSION

In summary, the present invention provides a system to manage pandemic health risk by and through a web accessible app having a user interface that runs on a mobile device; a voluntary participant can register to use the pandemic app; and a trace contact is identified in the pandemic app by the participant through the user interface.

The foregoing description of the preferred embodiments of the invention has been presented for the purposes of illustration and description. It is not intended to be exhaustive or to limit the invention to the precise form disclosed. Many modifications and variations are possible in light of the above teaching, it is intended that the scope of the invention are not, and need not be, limited by this detailed description, but by the claims and the equivalents to the claims which relate to the present invention. Use of punctuation and any articles “a” or “the” in reference to matter claimed shall be construed broadly to uphold the appended claims and equivalents thereto. This specification shall be construed broadly to uphold the claims and equivalents thereto, as set forth by the claims appended hereto.

What is claimed is:

1. A system to manage pandemic health risk stored on a non-transitory computer-readable storage medium, comprising:
a pandemic app having a user interface that is configured for use on a mobile device;
a voluntary participant can register to use the pandemic app; and
a trace contact is identified in the pandemic app by the participant through the user interface.

2. the system of claim 1, further comprising:
trace contact is identified within a first tier of trace contacts.

3. the system of claim 2, further comprising:
a first-tier trace contact is identified.

4. the system of claim 3, further comprising:
a participant can designate a trace contact with whom the participant had recent contact in a tracing disclosure record.

5. the system of claim 4, further comprising:
the user interface provides a geolocation of the participant relative to testing locations.

6. the system of claim 4, further comprising:
the participant inputs a voluntary self-quarantine report.

7. the system of claim 4, further comprising:
a contact tracer can identify the trace contact with whom the participant had recent contact in a tracing disclosure record.

8. the system of claim 7, further comprising:
a tracing disclosure record is stored in a relational database.

9. the system of claim 8, wherein:
the participant discloses one or more tracing contacts in the tracing disclosure record.

10. the system of claim 9, wherein:
a self-reported testing disclosure is made by the participant.

11. the system of claim 10, wherein:
a self-reported quarantine disclosure is made by the participant.

12. the system of claim 11, further comprising:
a participant's self-reported quarantine history report is sent to trace contacts of participant.

13. the system of claim 12, further comprising:
one or more social contacts of the participant are selected from: social media connections, email contacts, and phone contacts.

14. the system of claim 13, further comprising:
a medical history is associated with a participant.

15. the system of claim 14, further comprising:
a second tier of trace contacts associated with the person of interest through the first-tier trace contact.

16. the system of claim 15, further comprising:
the trace contact can designate voluntary self-quarantine in response to a disclosure of self-quarantine.

17. the system of claim 16, further comprising:
a timer is associated with a first time of reporting to expire at the conclusion of a known duration.

18. the system of claim 17, further comprising:
the timer is set to at least two weeks from the date of contact with a person of interest.

19. the system of claim 18, further comprising:
an automated electronic message is sent to a first tier of contacts associated with the participant.

20. the system of claim 19, further comprising:
the automated electronic message advises the trace contact to self-quarantine.

21. the system of claim 19, further comprising:
an order to send test kits to the first tier of contacts paid for by a sponsor.

22. the system of claim 19, further comprising:
an order to send test kits to the first tier of contacts paid for by a governmental agency.

23. the system of claim 19, further comprising:
an order to send test kits to the first tier of contacts paid for by a healthcare insurance provider.

24. the system of claim 19, further comprising:
the user interface provides nearby test locations near the participant wherein the participant can choose a selected test location.

25. the system of claim 19, further comprising:
the user interface provides the participant with a scheduled testing date, time and testing location based on the selected test location.

26. the system of claim 19, further comprising:
a timed follow-up message is sent to the trace contact at an end time of the contagious period for follow-up and second tier trace contacts associated with the first-tier trace contact.

27. the system of claim 19, further comprising:
a trace contact can initiate an order for testing kit to be shipped to the trace contact.

28. the system of claim 19, further comprising:
a certificate of quarantine completion can be generated via a certificate server based on a blockchain.

29. the system of claim 19, further comprising:
the certificate of quarantine completion can be confirmed as to authenticity via the blockchain.

30. the system of claim 19, further comprising:
a blockchain-based message is sent to the trace contact showing an anonymous sender.
